(19) United States
(12) Patent Application Publication
(10) Pub. No.: US 2020/0237462 A1
(43) Pub. Date: Jul. 30, 2020

(54) Surgical Safety Devices and Methods

(71) Applicant: Neuromuscular Surgical LLC, East Falmouth, MA (US)

(72) Inventors: James Keith Bleiler, East Falmouth, MA (US); Brett Zubiate, Duxbury, MA (US)

(21) Appl. No.: 16/652,967

(22) PCT Filed: Oct. 1, 2018
(86) PCT No.: PCT/US18/53809

§ 371 Date: Apr. 1, 2020

Related U.S. Application Data
(63) Continuation-in-part of application No. 15/721,938, filed on Oct. 1, 2017.

(51) Int. Cl. A61B 34/00; A61B 90/00; A61B 18/12

(52) U.S. Cl. CPC A61B 34/70; A61B 90/03; A61B 2018/00702; A61B 90/08; A61B 2562/0219; A61B 18/1206

(57) ABSTRACT

A surgical safety system for use in connection with a powered surgical instrument can be a standalone device or can be incorporated into existing electrosurgical instruments and generators. The system employs a controller connected to one or more sensors that are monitoring an aspect of a patient undergoing surgery. The controller is also in electrical communication with the powered surgical instrument and is configured to disable the instrument if movement or impending movement is detected.

### CROSS-REFERENCE TO RELATED APPLICATIONS

This patent application is an international application claiming priority to U.S. Non-Provisional patent application Ser. No. 15/721,938, filed Oct. 01, 2017.

### FIELD OF INVENTION

The present disclosure relates to a surgical safety system, apparatus or assembly, and method that employs sensors to detect patient movement or impending movement during a surgical procedure in order to prevent unnecessary injury. More specifically, the present disclosure is directed to surgical safety devices and methods that are particularly adapted for electrosurgical procedures that use instruments that employ an electrosurgical generator to apply current to a patient's tissue.

### BACKGROUND OF INVENTION

Modern surgical equipment and techniques often require a surgeon to perform surgical operations or procedures that require some type of energy to be delivered to the body of the patient in order to cut, coagulate, dissect, dessicate, or fulgurate tissue and is usually preferred because it is quick and minimizes bleeding. This use of energy in the operating room can, however, cause unintended consequences.

The use of electrically powered instruments, for example, may induce patient movement via unintended nerve or muscle stimulation. These instruments can cause irreversible injury when they are misaligned, misapplied, or inappropriately jarred by the unexpected movement of the patient's body during a procedure.

In the diagnosis and initial treatment of bladder cancer, for example, surgical procedures such as transurethral resection (TUR) are common. The aim of TUR is to remove all visible lesions and part of the underlying muscle tissue. Unfortunately, the obturator nerve passes close to the bladder wall and, if stimulated by the electrical current transmitted by an electrosurgically operated resectoscope during the removal of any lesions, can cause the patient's leg to jerk involuntarily, quickly, and unexpectedly. This is commonly referred to as obturator reflex, and it creates a substantial risk of bladder perforation, deep bladder wall tissue injury, nearby organ injury, or some other unintended injury to the patient, such as acute vascular injury of the pelvic vessels, which can be life-threatening.

Power to the resectoscope is routinely voluntarily activated by depressing a foot pedal or depressing a hand switch. Because it is nearly impossible to observe this reflexive movement before the sudden violent leg movement occurs, it is not possible to manually deactivate the instrument before it causes damage because human reaction time is too slow.

Traditionally, surgeons have used various techniques, including nerve blocks and induced paralysis, to address such problems. However, these techniques increase operating room time and associated cost and have additional potential complications. Moreover, they are usually implemented only after a reflexive incident and unintended tissue damage has already occurred.

Once a surgeon orders nerve blocks or induces paralysis, an anesthesiologist will often have to engage in more intensive airway management (e.g., intubation rather than laryngeal mask ventilation) as well as more intensive management of blood pressure. Medications to induce paralysis have a comprehensive list of potential complications that are well known but essentially will result in, at best, a longer recovery time for the patient and at worst, additional complication or adverse reactions.

Of course, electrosurgical devices are used in many surgical procedures beyond the diagnosis and treatment of bladder cancer. These devices are also used in procedures in fields such as gynecology, dermatology, cardiology, and orthopedics, among others.

The risks associated with sudden movements by patients in close proximity to electrosurgically powered surgical instruments are great. Human reaction time is insufficient, in many cases, to avoid injury and there are currently no safety systems or devices available to minimize these risks and/or to prevent these unnecessary injuries.

### SUMMARY OF INVENTION

The present disclosure is directed to a medical surgery safety methodology, systems, and assemblies that can be used in connection with an electrosurgically powered surgical instrument. In certain embodiments, the medical surgery safety system includes one or more electromyography skin surface electrodes for detecting electrical activity in a patient's muscle in response to voluntary or induced stimulation of the nerve. A controller and/or processor receives information from the electromyography sensor and disrupts power or control signals to the electrosurgical instrument being used. In certain embodiments, the controller deactivates the power to the surgical instrument after communicating with the electromyography sensor and detecting electrical activity in response to stimulation of a monitored muscle by the nerve.

Another embodiment taught by the present disclosure includes a medical surgery safety system that includes an inertial sensor such as an accelerometer or gyroscope to detect movement of a limb, whether in response to stimulation of the muscle group moving that limb by the nerve or by an external force such as an inadvertent nudge of the limb by a member of the surgical team. As with the EMG sensor, the controller and/or processor disrupts control or power to the electrosurgical instrument when certain conditions are detected.

Further embodiments include monitoring systems and devices that detect movement of a monitored limb such as a video imaging system used in conjunction with fiducial markers, 2D imaging, 3D stereoscopic imaging, and electromagnetic tracking of limbs. In the case of each of these systems, detection of movement would trigger an interruption in the control or power to the electrosurgical instrument.

In embodiments, the medical surgery safety system can be a standalone unit placed electrically between an electrosurgical generator and a hand piece with which the electrical current is applied by a surgeon to apply an electrical current to the patient's tissue to achieve a surgical goal. In another embodiment, the safety system can be incorporated within the control unit of a handpiece. In still another embodiment, the safety system can be incorporated within an actuation mechanism, such as a foot pedal, related to the handpiece. In a further embodiment, the safety system could be incorporated into the electrosurgical generator itself.

In embodiments, the medical surgery safety system can optionally include a display device for displaying information relating to the one or more motion prediction or motion detection sensors, power on or off, cautery safe or hot, and other metrics useful to a surgical team. In further embodiments, the controller can be a microcontroller and can connect to one or more sensors that are monitoring for potential or actual movement of a limb in an undesired way.

The medical surgery safety device can include a USB Interface for communicating with another device. The medical surgery safety device can connect to and be powered by a power supply selected from the group consisting of any reasonable medical grade power supply including, but not limited to, AC or DC power, a battery pack or a USB interface.

### BRIEF DESCRIPTION OF THE DRAWINGS

The accompanying drawings, which are incorporated in and form a part of the specification, illustrate the embodiments of the invention and together with the written description serve to explain the principles, characteristics, and features of the invention. In the drawings:

FIGS. 1 is a plan view of a typical prior art operating room illustrating the placement and arrangement of an electrosurgical generator, along with relevant accessories.

FIG. 2 is a plan view of an operating room with a stand-alone embodiment of a safety appliance employed.

FIG. 3 is a plan view of an operating room with an embodiment of the presently disclosed safety component incorporated into an electrosurgical generator.

FIG. 4 is a plan view of an operating room with an embodiment of the presently disclosed safety component incorporated into a footswitch.

FIG. 5 is a plan view of an operating room with an embodiment of the presently disclosed safety component incorporated into a handpiece.

FIG. 6 is a conceptual block diagram illustration of the main hardware and software components used in an embodiment of the stand-alone safety appliance.

FIG. 7 is a conceptual block diagram illustration of the main hardware and software components used in an embodiment of the stand-alone safety appliance designed for a TUR use case.

FIG. 8 is a flow chart demonstrating the work flow of the safety assembly described herein.

### DETAILED DESCRIPTION OF THE INVENTION

This disclosure is directed to systems and methods for implementing a surgical safety device that uses sensors to detect patient movements or potential movements during surgical operations and enables a precautionary response to occur faster than the surgeon would be able to react. In the following description, for purposes of explanation, numerous specific details are set forth in order to provide a thorough understanding of example embodiments. It will be evident to one skilled in the art, however, that embodiments can be practiced without these specific details. The description is not to be taken in a limiting sense but is made merely for the purpose of illustrating the general principles of the invention, since the scope of the invention is best defined by the claims included herein.

In certain embodiments, surgical safety devices constructed in a manner consistent with the present disclosure use sensors such as EMG sensors, accelerometers, and gyroscopes, among others, to anticipate or detect unexpected bodily movements in patients during surgical procedures. In embodiments, the devices are part of a safety system that can optionally use a bilateral sensor array to measure activity of both of a patient's extremities in either an absolute or relative way. Upon anticipation or detection of any undesired movements or anticipated movements, the disclosed safety device and system will shut off power to certain electrical surgical instruments to protect the patient from accidental harm.

In certain embodiments, the disclosed surgical safety system uses EMG detection sensors to measure muscle response or electrical activity in a muscle in response to nerve stimulation. EMG detection sensors include one or more small needles or electrodes that can be inserted into the skin only, through the skin into the muscle, or can take the form of a disposable gelled ECG/EMG surface sticker monitoring electrode to detect electrical activity. The sensors can use the signal detected by any of these sensors to send signals to controllers, computing systems, computing devices, and/or display devices for analysis. In response to appropriate signals based on data from the sensors, such devices can be used to shut off, override, or otherwise control surgical devices to prevent them from injuring patients when such movements occur.

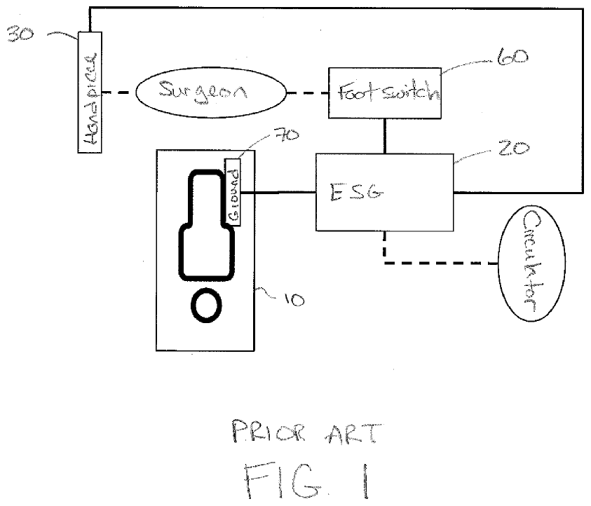

FIG. 1 depicts a typical medical surgery operating room (OR) environment. The medical equipment in the OR often includes at least an operating table 10, an electrosurgical generator (ESG) 20, and a powered tool handpiece 30. Typically, power to the handpiece 30 is controlled by the surgeon using a footswitch 60.

In a typical procedure that uses an ESG 20, a surgeon uses at least the handpiece 30 to perform the procedure. An anesthesiologist keeps the patient appropriately sedated and a scrub nurse is available to assist the surgeon. The OR can include other medical professionals, such as a circulator, and medical equipment, depending on the particular type of surgical procedure. Note that this prior art setup does not address the problem of avoiding injury to the patient due to unexpected movement of the patient during use of the handpiece.

Electrosurgery, as discussed herein, employs electric current from the ESG 20 in the form of radio frequency (RF) current that is applied to the patient's tissue through the handpiece 30. The current can be applied using bipolar or monopolar handpieces, or resection loops. Bipolar handpieces are used for situations where grippers, forceps, or resection loops are desired for a particular procedure and the current passes from one side of the contacts and leaves the tissue from the other. For monopolar use, as is described and illustrated herein, the handpiece 30 has a pointed, hooked, or bladed tip to concentrate the current flow in a specific area. Because there is only one point of contact, the circuit must be completed through the use of a wide ground pad 70 that is attached to the patient.

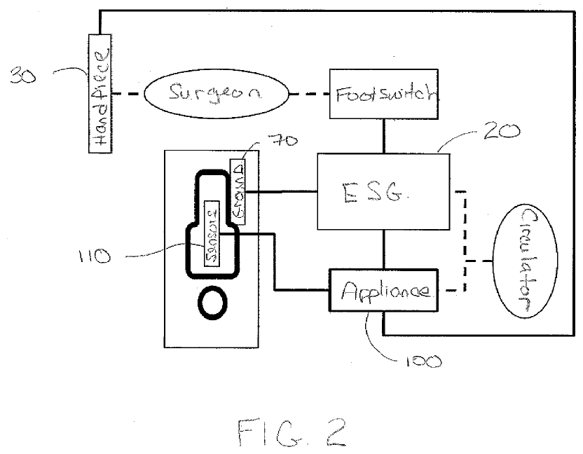

FIG. 2 illustrates an OR environment with a system constructed in accordance with an embodiment of the present invention installed. As illustrated, a stand-alone surgical safety appliance 100 is communicatively placed between the ESG 20 and the handpiece 30 so that it can control the signals or power being delivered to the handpiece 30. The surgical safety appliance 100 functions as a protective interface that employs sensors to monitor for motion or impending motion of a patient's limb and controls the power supply to the handpiece 30. In the event of an unexpected firing of a muscle or movement of the limb, embodiments of the surgical safety appliance 100 deactivate or interrupt the power supply to the handpiece 30 to prevent injury to the patient.

In embodiments, the surgical safety appliance 100 monitors sensors 110 that are attached to at least the relevant limb or body part of the patient to provide motion or other relevant information about the patient. In certain embodiments, those sensors 110 include one or more EMG sensors, which can provide a very fast response time on the order of less than twenty-five (25) milliseconds. In certain other embodiments, sensors 110 can include one or more inertial sensors, such as accelerometers or gyroscopic sensors, which provide a response in the neighborhood of between twenty-five (25) and one hundred (100) milliseconds. Sensors 110 are sensitive to electromagnetic disturbances, however, and electrical cables should be shielded, such as with a Faraday cage, whenever they are used. It should be noted that EMG sensors are will detect impending movement, but not movement where there is not muscle activity, such as when a patient's limb is bumped or when a surgical team member is moving a patient's limb. For this reason, it is within the scope of the present disclosure to have more than one type of sensor employed with the system.

In still other embodiments, a camera may be used in the operating room, the camera capable of detecting fiducial markers connected to the patient's body (such as to the patient's bone or skin) such that any unexpected movement can be detected and reported back to a monitoring system on the appliance or to some system in communication with the appliance. The response time for this last type of sensing system is between one hundred (100) and three hundred (300) milliseconds.

There are many types of procedures for which systems and methods of the present disclosure are useful. For purposes of further explanation, the use case of a transurethral resection (TUR) will be discussed due to the chance of injury when electrosurgical instruments are used, as previously discussed. In an embodiment directed toward reducing the chance for injury to a patient undergoing TUR, the sensors 110 are accelerometers that are attached to the patient's thighs or as close to the knees as possible. The accelerometers detect movement of the legs or other monitored body parts, such as might occur from accidentally bumping the patient in the OR or causing a reflex action to occur. Upon movement detection based on an appropriate reading from one of the accelerometers, the appliance 100 cuts power or otherwise disables the handpiece 30.

In another embodiment, the sensors 110 are EMG sensors that are placed on the appropriate leg muscle to detect unexpected electrical activity that might occur prior to actual movement of the limb, such as when a surgeon unintentionally energizes the obturator nerve, which could cause the surgical safety appliance 100 to disable the handpiece 30 or otherwise cause the electrosurgical generator 20 to cease sending power or signals to the handpiece 30. In this embodiment, a baseline level of average electrical activity can be captured prior to the surgeon beginning the procedure so that an elevation in activity preceding movement can be detected and action can be taken prior to actual movement.

It will be apparent to those of skill in the art that the appliance 100 could also be placed between the power source and the ESG 20. In that location, upon identification of movement or intended movement, the appliance 100 either cuts power or otherwise stops the delivery of power to the handpiece 30.

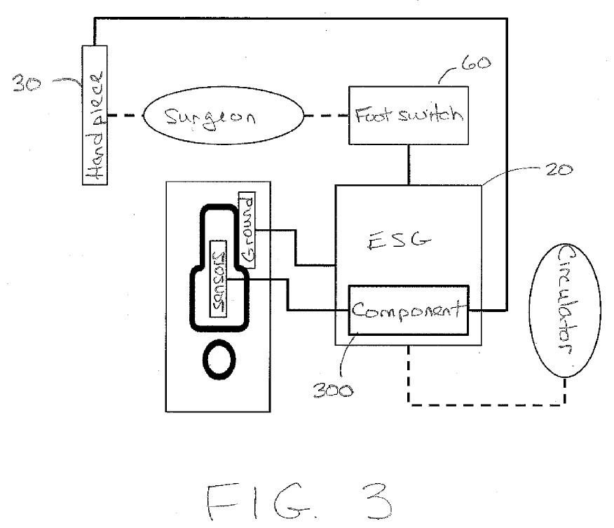

Referring now to FIG. 3, an additional embodiment is illustrated wherein the features of the surgical safety appliance 100 are incorporated as a surgical safety component 300 into the ESG. The component 300 operates in all material respects in the same way as the standalone appliance 100 described previously, except it is incorporated into the ESG 20 as an OEM product.

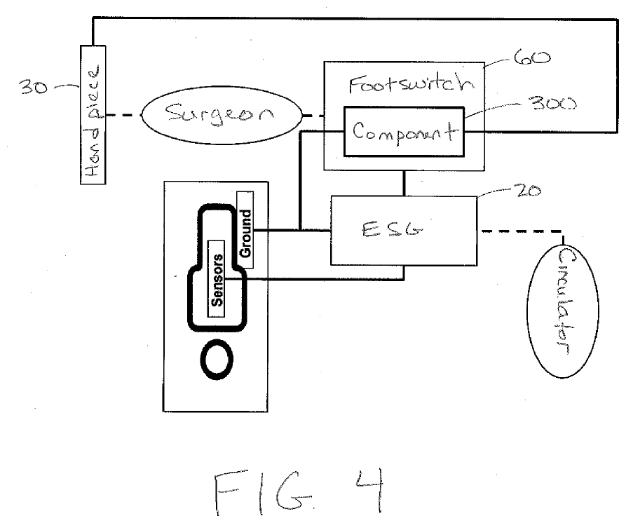

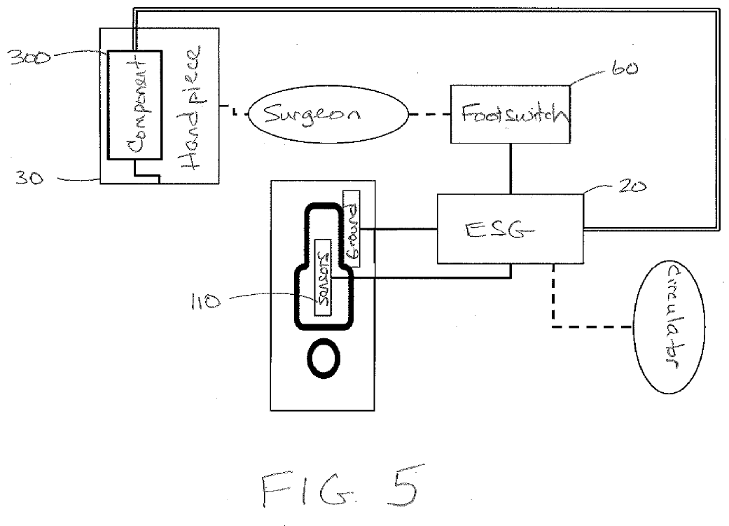

FIGS. 4 and 5 demonstrate additional embodiments wherein a surgical safety component 300 is incorporated into the foot switch or the handpiece. In embodiments, the ESG 20 can be modified to pass data from the sensors 110 through to the component 300 embedded in the footswitch 60 or handpiece 30 depending on the embodiment. In alternative embodiments, data from the sensors can be delivered to the component by direct wire or wirelessly, without involving the ESG 20.

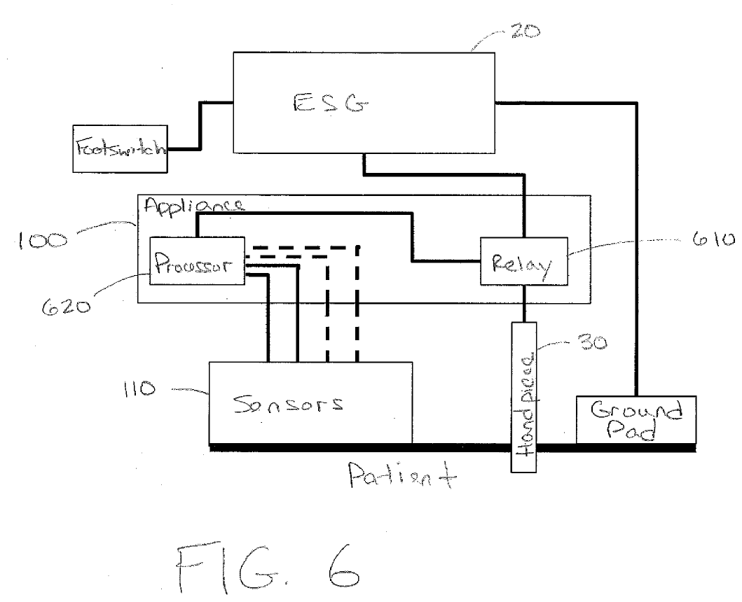

FIG. 6 depicts a block diagram of a surgical safety appliance 100 that can implement features of the disclosed invention. In certain embodiments, the surgical safety appliance 100 contains a processor 620 communicatively connected to sensors 110 that are monitoring a patient. In embodiments, the processor 620 can comprise a programmable microcontroller that can process multiple channels of signals and may also comprise a memory storage facility that stores threshold values the processor 620 can compare sensor measurements to in order to determine whether action should be taken, such as interrupting power to the handpiece 30. In embodiments, the threshold values may be based on pre-determined absolute values, such as in the case of inertial sensors, or they may be relative values that depend on a measured average, such as an amount of electrical activity in a muscle used as a baseline with a certain percentage increase over the baseline constituting an actionable signal.

In an embodiment, neural networks are used to analyze historical signals from the sensors and determine when movement may occur, whereupon the relay will be opened and operation of the handpiece stopped.

The processor 620 is also communicatively connected to a relay 610 that electrically sits between the ESG 20 and the handpiece 30 to allow or block electricity and/or information in response to the evaluation of sensor signals. In embodiments, the processor is controlled by software or firmware. In certain embodiments, processor 620 may also be a separate, dedicated processor for handling data or signals from the one or more sensors and is separate from any processors incorporated into appliance 100 or ESG 20

In further embodiments, the processor includes redundant processor architecture to mitigate the risk of software becoming corrupted. There may be two processors that work together with a first processor monitoring the inputs from the sensors and double-checking performance and a second processor controlling the relay and possibly a display associated with the surgical safety appliance, if separate from other devices or associated with the ESG, handpiece or footswitch if integrated therein. If the two processors do not agree, a cross-cheek failure will cause power to be interrupted to the handpiece. An example of this type of processor is the Hercules Safety MCU from Texas Instruments.

In another embodiment, the processor may comprise multiple processors working independently and performing the same calculations for all core functions. A fault occurs and power to the handpiece is interrupted if all of the processors don't agree.

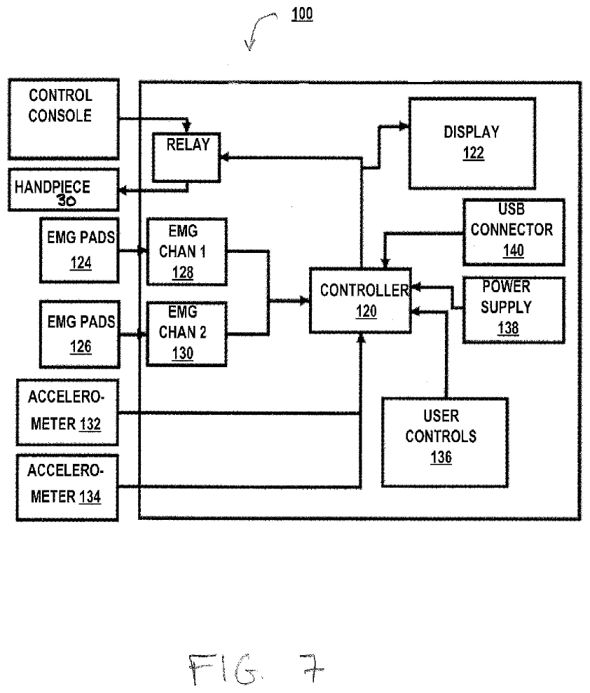

FIG. 7 illustrates an alternative embodiment of an appliance 100 configured to perform cauterization, which can be used during bladder surgery, prostate surgery, transurethral resection procedures, and many other surgical procedures. In this embodiment, the appliance 100 can be a cauterization system that includes a control console 710, a handpiece 30, such as a cautery instrument, and a surgical safety appliance 100 sitting electrically in between the control console 710 and the handpiece 30. The surgical safety appliance 100 functions as a protective interface that, upon receiving information at the controller 120 about unexpected motion or potential motion of a patient's limb from one or more sensors 124, 126, 132, 134, quickly employs the relay 610 to cut power or otherwise deactivate the handpiece 30 to prevent unintended cutting of tissue.

In certain embodiments, the appliance 100 cuts power to the handpiece 30 within a fraction of a second. In further embodiments, the appliance 100 cuts power to the handpiece 30 within 0.2 seconds. In this way, the surgical safety appliance 100 functions as a safety interface between the control console 710 and the handpiece 30. It is also within the scope of the present disclosure for the functionality of the appliance to be incorporated into an existing EMG device such as an EMG monitor. In such an embodiment, the EMG monitor readings would be delivered to the appliance in the event of an unexpected or anomalous reading whereby the ESG would be disabled, powered down or otherwise controlled. By way of further non-limiting explanation regarding this embodiment, the appliance 100 functionality would be incorporate into the EMG Monitor and provision would be made for the EMG monitor to be electronically communicative with the handpiece 30 and the ESG and/or control console 710.

By way of further non-limiting example, a tumor to be resected on the right side of the bladder could potentially cause right-sided obturator reflex and thus the right leg would be monitored for movement or muscle recruitment. The one or more EMG pads 124, 126 enclose EMG sensors that connect to the controller 120 in the device enclosure 116 through one or more EMG boards 128, 130. In certain embodiments, the EMG board 128 is a single channel EMG sensor processing device that can capture EMG sensor data that corresponds to the movement of a patient. In other embodiments, there can be multiple channel EMG boards without deviating from the invention.

In embodiments, the controller 120 can monitor one or more EMG pads 124, 126 and one or more EMG boards 128, 130 to detect actual or imminent leg movements. The EMG sensors within the EMG pads 124, 126 can be attached to a patient's legs to allow the controller 120 to monitor the adductor muscles therein. The controller 120 can use the sensors to detect electrical activity in the muscle indicating that a patient is about to move, even when the patient is unconscious. In certain embodiments, the controller 120 receives signals obtained by direct sensing of a muscle and/or nerve group by the monitoring electrodes in the EMG pad or pads 124, 126. In certain embodiments, the EMG pads are comprised of cutaneous gelled monitoring electrodes, although other types of sensors can be used without deviating from the invention.

In certain embodiments, the EMG pads 124, 126 record background or baseline electrical activity within a muscle. In the event of a spike in activity, which may indicate imminent movement of the patient's limb, the controller 120 cuts power to the handpiece 112 to avoid unintended injury. In further embodiments, detection of activity in the range of five to ten percent above the baseline of activity will cause the controller 120 to cut power. In still further embodiments, detection of electrical activity in the range of seven percent above a baseline of activity will trigger a cut in power to the handpiece 112. The controller 120 may also be biased to cut power in the case that the sensor data is inconclusive or unavailable. In another non-limiting example, the controller 120 monitors the sensor data and, should interpretation of that sensor data be inconclusive, the system or controller 120 will bias to failsafe. As used herein, the term failsafe is intended to mean the system or controller 120 cuts power to the handpiece 112 to protect the patient. In still another embodiment, if a measured value reported from sensor data, when taking uncertainty or error into account, could exceed a predetermined threshold, the system or controller 120 will bias to failsafe. In certain other embodiments, if trends, based on sample data, predict the threshold value for failsafe is likely to be breached, the system or controller 120 will bias to failsafe.

Optionally, the surgical safety device 114 can be connected to one or more inertial sensors 132, 134, such as accelerometers or gyroscopes. In embodiments, these inertial sensors 132, 134 can be positioned on a patient's leg on or adjacent to the kneecap to detect movement of the leg. Alternatively, the accelerometers can detect changes in velocity and acceleration of the limb to which they are attached. If motion is detected, the one or more accelerometers send signals that include information relating to the movement to the controller 120 within the device enclosure 116.

In embodiments, the controller 120, in turn, monitors the inertial sensors 132, 134 to detect unexpected limb movements in patients. The controller 120 can use the information either separately, or in conjunction with, the sensors in the EMG pads 124, 126 to detect or predict impending or actual limb movement. Upon detection of such sensor readings, the controller 120 can interrupt power to the handpiece 112 through the use of a relay or other similar mechanism as known to those of skill in the art.

The controller 120 can be implemented by software, hardware, firmware or a combination thereof. For example, the controller 120 can include components implemented by computer-executable instructions that are stored on one or more computer-readable storage media and that are executed to perform various steps, methods, and/or functionality in accordance with aspects of the described subject matter.

The controller 120 can store sensor readings in memory to analyze the sensor readings from the one or more accelerometers 132, 134 and/or the sensors in the one or more EMG pads 124, 126 for indications or precursors of patient movement. The controller 120 can implement one or more algorithms to detect, identify, or predict patient movements. In some embodiments, the algorithm or algorithms can account for factors, such as sensor placement variation. Other algorithms may allow certain “slow” intentional movement such as surgeon repositioning patient and only disconnect power upon the occurrence of “fast” or sudden movement. In still other embodiments, the controller 120 can communicate with the display device 122 to enable the display of output relating to the accelerometers 132, 134 and/or the sensors in the EMG pads 124, 126. The display 122 may alternatively simply be a light or even a noise that notifies the user of a fault condition. As mentioned in relation to FIG. 6, neural networks may also be employed to predict future movement based on past activity.

In embodiments, the EMG sensors in the EMG pads 124, 126 can be sensitive to variation in electrode placement, so that the controller 120 can store and compare sensor readings to an average of a number of samples over a predetermined time period, such as five seconds or less, to account for such variations. The controller 120 can be set to activate a protective circuit, in embodiments, this may actuate a relay 118 when sensor readings exceed the windowed average by a predefined threshold, such as seven percent, by way of example.

In embodiments, the controller 120 can be connected to user controls 136 that are housed within or on the device enclosure 116. The user controls 136 can include one or more buttons for powering up the surgical safety device 114 and one or more buttons for resetting the surgical safety device 114. In this exemplary embodiment, the controller 120 must wait for a reset signal from the user controls 136 before the relay 118 can reconnect the control console 110 to the handpiece 112. In embodiments, there may also be user controls 136 for changing the sensitivity of the controller such that it may cut power upon detection of either more or less electrical impulse activity. Where accelerometers are used, the user controls 136 may offer the user the ability to make the controller 120 more or less sensitive to movement of a limb detected by an accelerometer.

The controller 120 can be powered by a power supply 138 or by a USB connector 140. The power supply 138 can be an internal power source or an external power source. The USB connector 140 can connect to an external computer system or computing device (not shown).

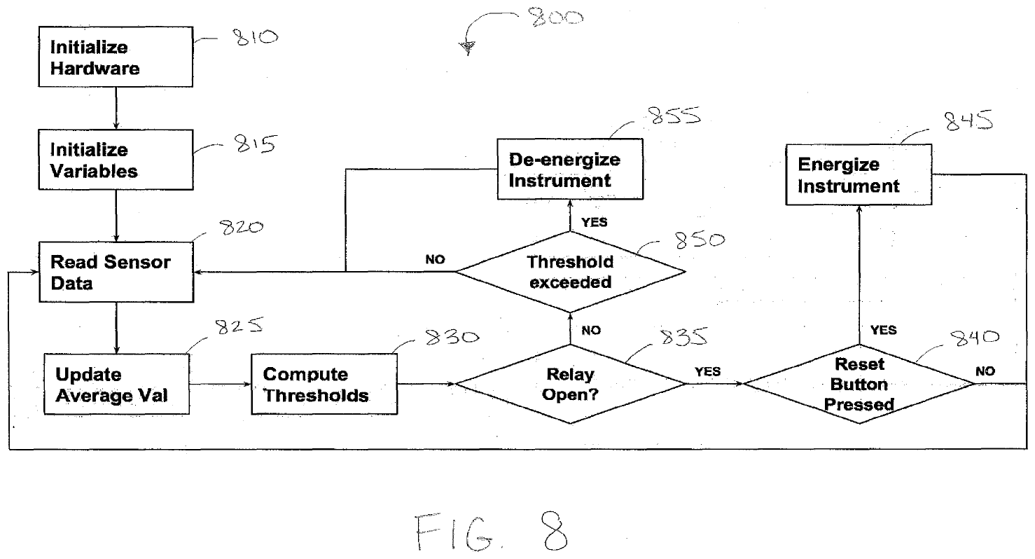

FIG. 8 demonstrates a procedural safety work flow 800 in accordance with an embodiment of the surgical safety device of the present invention whereby unintended injury secondary to the use of electrosurgical instruments such as has been described above can be avoided. For purposes of this example flow chart, the use of EMG sensors is assumed, but other sensors, such as inertial sensors, can also be used with only minor changes to the work flow.

At box 810, the processor or controller initializes the hardware to be used for the safety system to operate properly. Typically, this includes the inertial and electrical sensors that will be used during the surgical procedure. Next, at box 815, the variables, such as sensor readings, are initialized or tared to ensure accurate readings during the procedure.

At box 820, sensor data is recorded and an average value for each installed sensor is established with the initial reading at box 825. In certain embodiments, action threshold values are then calculated as a percentage increase over the established average value at box 830. In other embodiments, the action threshold values are pre-established and retrieved from memory for this step.

Once the thresholds are computed or otherwise established, the handpiece or instrument is ready to be used. At box 835, the system determines whether the relay is open or closed. An open relay indicates the instrument is not operational and the system will do nothing further until the reset button is pressed or the system is otherwise prepared for use at box 840 and the surgeon may energize the instrument at box 845. This may occur through the use of a foot pedal or trigger switch as described previously.

During the time the relay is closed and the instrument is energized and ready for use or is actually in use, the system will continue to cycle through boxes 820-835 so long as the action threshold is not exceeded, which determination is made at box 850. If the system determines that the action threshold has been exceeded, which would indicate impending movement or actual movement, the relay is immediately opened, which de-energizes the instrument as in box 855. After the movement of the patient is over and the source of the movement has been identified and rectified, the system re-starts at box 820 and the reset button is pressed, which re-energizes the instrument and the procedure continues without injury.

While various illustrative embodiments incorporating the principles of the present teachings have been disclosed, the present teachings are not limited to the disclosed embodiments. Instead, this application is intended to cover any variations, uses, or adaptations of the present teachings and use its general principles

I claim:

1. A surgical safety system for detecting unexpected patient activity during a procedure involving a powered surgical instrument, the surgical safety system comprising:
a controller in electrical communication with the powered surgical instrument; and
one or more sensors monitoring the patient for unexpected activity, the one or more sensors in electrical communication with the controller;
whereby, upon communication to the controller of the detection of unexpected activity by the one or more sensors, the controller disables the powered surgical instrument.

2. The surgical safety system of claim 1, wherein the one or more sensors are electromyographic sensors for detecting muscle activity that indicates imminent movement.

3. The surgical safety system of claim 1, wherein the one or more sensors are one or more of accelerometers and gyroscopes for detecting movement.

4. The surgical safety system of claim 1, further comprising a display device for displaying information relating to unexpected movements.

5. The surgical safety system of claim 1, further comprising one or a plurality of electromyography boards in electrical communication with the controller, wherein each board is configured to transmit EMG sensor data that corresponds to the movement of a patient to the controller.

6. The surgical safety system of claim 1 further comprising a powered instrument control console for supplying power to the powered surgical instrument, the control console in electrical communication with the controller, whereby the controller can shut off the power to the powered surgical instrument if unexpected activity is detected.

7. The surgical safety system of claim 6, wherein the system is incorporated into the
instrument control console.

8. The surgical safety system of claim 1 wherein the powered surgical instrument further comprises a power source and the controller is electrically positioned in between the powered surgical instrument and the power source.

9. The surgical safety system of claim 1 wherein the powered surgical instrument is controlled by a switch and the surgical safety system is incorporated into the switch.

10. The surgical safety system of claim 1, further comprising a data communication port for exchanging data with the controller.

11. The surgical safety system of claim 1, wherein the system is incorporated into the powered surgical instrument.

12. A method for controlling a surgical instrument to prevent injury to a patient, the method comprising the steps of:
detecting unexpected activity; and
deactivating the surgical instrument in response to the unexpected activity.

13. The method of claim 12, wherein the step of detecting unexpected activity is performed with one or more of electromyography sensors, accelerometers, gyroscopes, cameras, and electromagnetic sensors.

14. The method of claim 12, further comprising communicating to a controller that unexpected activity has been detected and deactivating the surgical instrument in response.

15. The method of claim 12, further comprising displaying output relating to the unexpected activity.

16. An electrosurgery safety system comprising:
a powered surgical instrument;
an electrosurgical generator with a control console for controlling and delivering power to the powered surgical instrument; and
a safety controller electrically situated between the electrosurgical generator and the powered surgical instrument, the safety controller having at least one sensor to monitor for unexpected activity;
wherein the safety controller deactivates the surgical instrument after receiving an indication from the at least one sensor that indicates that unexpected activity has been detected.

17. The electrosurgery safety system of claim 16, wherein the safety controller comprises one or a plurality of accelerometers for detecting unexpected movement.

18. The electrosurgery safety system of claim 16, wherein the safety controller comprises one or a plurality of electromyography sensors for detecting unexpected electrical activity in a muscle.

19. The electrosurgery safety system of claim 16, wherein the safety controller comprises one or a plurality of electromyography sensors and one or a plurality of accelerometers.

20. The electrosurgery safety system of claim 16, wherein the safety controller comprises one or more of electromyography sensors, accelerometers, gyroscopes, cameras, and electromagnetic sensors.
